(19) United States
(12) Patent Application Publication
(10) Pub. No.: US 2022/0257872 A1
(43) Pub. Date: Aug. 18, 2022

(54) PROTECTIVE CAP FOR ATTACHMENT TO A NEEDLE STICK PROTECTION DEVICE FOR A SYRINGE, SYRINGE SAFETY SYSTEM AND SYRINGE SYSTEM

(71) Applicant: Schreiner Group GmbH & Co. KG, Oberschleissheim (DE)

(72) Inventors: Robert GEISSLER, Muenchen (DE); Andreas HOFENAUER, Germering (DE); Stefan KRAUSS, Muenchen (DE); Mathias FREY, Oberschleissheim (DE); Benny GOETZE, Muenchen (DE); Raphael BORY, Freising (DE)

(73) Assignee: Schreiner Group GmbH & Co. KG, Oberschleissheim (DE)

(21) Appl. No.: 17/626,191

(22) PCT Filed: Jul. 13, 2020
(86) PCT No.: PCT/EP2020/069748

§ 371 Date: Jan. 11, 2022

(30) Foreign Application Priority Data
Jul. 12, 2019 (DE) 10 2019 119 011.8

(51) Int. Cl. A61M 5/32; A61M 5/50

(52) U.S. Cl. CPC A61M 5/3202; A61M 2205/6054; A61M 5/5086

(57) ABSTRACT

A protective cap for attachment to a needle stick protection device (20) for a syringe includes a sleeve-shaped cap body configured to enclose a needle guard of the syringe. The protective cap further includes at least one coupling member coupled to the cap body and configured to couple the cap body to an outer housing of the needle stick protection device and to secure the protective cap to the needle stick protection device.

The present invention relates to a protective cap for attachment to a needle stick protection device for a syringe, which provides reliable protection for a syringe provided with the needle stick protection device in a simple and cost-effective manner. The invention further relates to a syringe safety system and a syringe system each comprising such a protective cap.

Conventional syringes usually have a needle guard which encloses the syringe needle and protects it from unwanted contact. Furthermore, needle stick protection devices are usually provided which are coupled to a syringe and are intended to provide reliable protection from the syringe needle after the syringe has been used. However, there is a possibility that when an impact occurs, such as when the syringe is dropped, a force may be applied to the needle guard and/or the needle stick protection device which may result in damage to the syringe needle, inadvertent triggering of the needle stick protection mechanism or release of the holding mechanism of the needle stick protection device, causing the syringe to slip out of the needle stick protection device.

It is an object underlying the present invention to contribute in a simple and cost-effective manner to a reliable protection for a syringe provided with a needle stick protection device.

The object is solved by the features of the independent patent claims. Advantageous developments are indicated in the respective dependent claims.

According to one aspect of the invention, a protective cap for attachment to a needle stick protection device for a syringe comprises a sleeve-shaped cap body adapted to enclose a needle guard of the syringe. The protective cap further comprises at least one coupling element coupled to the cap body and adapted to couple the cap body to an outer housing of the needle stick protection device and to fix the protective cap to the needle stick protection device.

By means of the described protective cap, a reliable protection for a syringe provided with the needle stick protection device can be realized in a simple and cost-effective manner. The protective cap works together with the needle stick protection device and contributes to a safe use of the syringe. The needle stick protection device is coupled to a syringe and serves to form a reliable protection against the syringe needle after a use of the syringe. For example, it comprises a retaining mechanism by which it is coupled to a syringe housing of the syringe and a trigger mechanism which provides protection from the syringe needle.

In particular, the protective cap serves to protect against unintentional triggering of the needle protection mechanism and against unintentional release of the holding mechanism of the needle stick protection device, in which the syringe can slip out of the needle stick protection device. By means of the protective cap, it can be prevented that a force, for example caused by the syringe falling down, does not act directly on the needle guard and thus also on the syringe, but is absorbed by the protective cap.

In addition, the protective cap protects the syringe needle, which is usually surrounded by a needle guard. The needle guard comprises, for example, an elastomer element which is placed over the syringe needle as a rubberised attachment. In addition, a plastic sleeve may be provided which surrounds the elastomer element and the syringe needle.

It is a finding in connection with the present invention that, for example, a needle guard of a pre-filled syringe with an inserted syringe needle, which are embedded in a needle stick protection device, may be susceptible to impact effects. Impact, for example dropping and hitting the syringe, exerts a force on the needle guard which may result in damage to the syringe needle, for example bending, damage to the grinding of the syringe needle due to relative movement of the elastomer of the needle guard relative to the syringe needle, or particle formation due to relative movement of the elastomer of the needle guard relative to the syringe needle. Further, an unintentional force may cause the needle guard mechanism to disengage or the needle guard retaining mechanism to disengage, causing the syringe to slip out of the needle guard.

The protective cap described contributes to additional protection of the syringe and in particular the syringe needle. It enables safe and reliable use of a syringe provided with a needle stick protection device. In addition, the protective cap enables the syringe to be sealed, thereby guaranteeing or implying that the syringe is intact. Furthermore, a proof of first opening can be realized by means of the protective cap, so that a first opening and thus a possible loss of sterility can be clearly indicated. The protective cap is made, for example, of plastic, can be manufactured inexpensively and provides reliable mechanical protection for the needle guard or the syringe needle of a syringe and can also establish a secure seal for the syringe.

According to a preferred embodiment, the protective cap comprises at least one cap arm coupled to the cap body. The coupling element is arranged or formed on the cap arm such that the cap body can be coupled to the outer housing of the needle stick protection device by means of the cap arm, and the protective cap can be attached to the needle stick protection device.

Preferably, the protective cap comprises at least a second cap arm which is connected to the cap body. Further provided is a further coupling element coupled to the second cap arm and configured to couple the cap body to the outer housing of the needle stick protection device and to secure the protective cap to the needle stick protection device. The cap arm or arms extend, for example, from the cap body towards the needle stick protection device, which typically surrounds a syringe cavity with the contents therein.

According to a further preferred embodiment of the protective cap, at least one coupling element is designed as an engagement element and is set up to engage and snap into a recess of the needle stick protection device. The recess of the needle stick protection device is, for example, a laterally arranged groove which extends longitudinally and is delimited in a predetermined manner by the outer housing of the needle stick protection device. An outer contour of the engagement element is preferably formed in coordination with a contour bounding the recess of the needle stick protection device. Preferably, two cap arms are provided, each with one or more engagement elements which can engage in corresponding recesses on the needle stick protection device.

According to a further embodiment of the protective cap, at least one coupling element is formed as a clip element and is adapted to clip around a portion of the needle stick protection device. The clip element has, for example, two gripping wings which, in an arranged state, rest in a gripping manner against a portion of the needle stick protection device.

According to a further embodiment of the protective cap, at least one coupling element is formed as an adhesive surface and is adapted to form an adhesive coupling with the outer housing of the needle stick protection device. In this way, the protective cap can be easily and reliably bonded to the needle stick protection device.

According to a further embodiment of the protective cap, at least one coupling element is formed as a label and is adapted to connect the cap body to the needle stick protection device. The label enables the protective cap to be bonded to the needle stick protection device in a simple and reliable manner. In addition, the label enables information to be applied by means of printing or inscription.

The label has, for example, a perforation along which the label can be severed in a predetermined manner so that a label section can be removed from the needle stick protection device together with the cap body. Another label section remains on the syringe or needle stick protection device, for example. The label thus also provides proof of opening by predetermined tearing along the perforation during an opening operation.

The protective cap can also be coupled to the needle stick protection device by means of several labels, which are formed in one or more layers. The label or labels may be aligned parallel to a longitudinal axis of the needle stick protection device or the protective cap, and may couple the needle stick protection device to the protective cap. Alternatively, the label or labels connecting the needle stick protection device to the protective cap may be oriented perpendicular to the longitudinal axis of the needle stick protection device and cover, for example, a perimeter of the needle stick protection device. The label may further be in the form of a label overlapping on itself and, once attached, may come to rest on itself.

A label used to attach the protective cap to the needle stick protection device may also be designed to cover as large a portion of the needle stick protection device as possible, thereby protecting the syringe and its contents from UV radiation or visible light. This can be set up, for example, by a foil from which the label is made having UV or light protection properties.

Alternatively or additionally, according to a further embodiment, the protective cap has a predetermined breaking point formed between the cap body and the first and/or the second cap arm, so that the protective cap can be severed in a predetermined manner and the cap body can be removed from the first and/or the second cap arm. Thus, proof of opening can also be provided by the protective cap breaking through at the predetermined breaking point during an opening operation, leaving one cap arm attached to the needle stick protection device.

According to further embodiment, the protective cap has an interlocking structure formed on an inner surface of the cap body and adapted to interlock or couple with an outer surface of the needle guard of a syringe when the protective cap is attached to the syringe. Alternatively or additionally, an interlocking structure may be formed on existing cap arms of the protective cap.

By means of the interlocking structure, the protective cap can mechanically connect to the needle guard, so that when the protective cap is removed from the syringe, the protective cap is removed together with the needle guard. In this way, when the syringe is opened, no additional work step is required to expose the syringe needle. Alternatively, the protective cap is configured mechanically decoupled from the needle guard to increase a level of protection in case of mechanical impact.

Further, the protective cap may have one or more penetrating recesses or openings formed on the cap body and/or on the cap arm or cap arms. Such a protective cap implements a protective sheath for the syringe needle with one or more viewing windows that allow visual inspection of the syringe or syringe needle.

According to a further embodiment, the protective cap has a predetermined weakening structure which is arranged in such a way that, with respect to a state arranged on the needle stick protection device, an irreversible geometrical change of the protective cap is formed during a removal process. The protective cap thus provides a broken seal that provides proof of opening. If, for example, the protective cap is removed for use of the syringe, the protective cap undergoes a defined change, for example by breaking or deformation, so that a renewed mechanical fixation to the needle stick protection device is not feasible when the protective cap is put back in place. Alternatively or additionally, such a seal break may be provided by one or more labels which are irreversibly destroyed when the protective cap is removed.

According to a further preferred embodiment of the protective cap, an outer contour of the protective cap is formed in coordination with an outer contour of the outer housing of the needle stick protection device, so that with respect to a state arranged on the needle stick protection device, a flush transition between the protective cap and the outer housing of the needle stick protection device can be established.

For example, the protective cap is applied to the outside of the needle guard of a syringe and is shaped such that its outer structure is flush with the outer structure of the needle guard in which the syringe is embedded. This ensures that the protective cap can be easily and reliably attached to the needle stick protection device, for example by one or more labels. Thus, edge or folding in a label is avoided or counteracted. Such a flush or contour-extending design of the protective cap contributes to a beneficial labelling capability.

The protective cap may further be configured to interlock with the needle stick protection device when attached to the syringe or needle guard, such that it is sufficiently tightly coupled to the needle stick protection device in a packaging process for application of a label to connect the needle stick protection device to the protective cap. Such interlocking is realized, for example, by the engagement element or elements that engage in recesses of the needle stick protection device or grip around edges of the needle stick protection device.

The interlocking of the protective cap with the needle stick protection device can be designed so firmly or stably that an additional fastening with a label is not necessary. In this case, an interlocking structure of the protective cap is adapted to standard needle stick protection devices, for example. Thus, it is not necessary to modify the needle stick protection device for the interlocking or mechanical coupling with the protective cap. Accordingly, needle stick protection devices available on the market can be additionally functionally extended with the protective cap without having to be modified themselves.

The interlocking or mechanical coupling of the protective cap with the needle stick protection device can be designed in such a way that no components of the protective cap remain on the needle stick protection device when the protective cap is removed, for example for use of the syringe. Alternatively, intended components may selectively remain on the needle stick protection device and provide a proof of first opening. In each case, however, the protective cap is designed in particular such that the function of the needle stick protection device is not impaired in any way. It is therefore not necessary to change, modify or adapt the needle stick protection device and its function to the protective cap.

If a force acts on the protective cap, for example due to the syringe falling and hitting a floor, this force is diverted to the needle stick protection device by the mechanical coupling, so that no force or a significantly reduced force acts on the needle guard and thus on the syringe needle, thus protecting it advantageously. Damage to the syringe needle as well as an unintentional triggering of the needle stick protection mechanism or a loosening of a holding mechanism of the needle stick protection device on the syringe, so that the syringe slips out of the needle stick protection device, can be avoided by means of the described protective cap.

The cap may further be configured and attached to the needle stick protection device such that it may be removed by a movement parallel to the longitudinal axis of the needle stick protection device or the protective cap, or perpendicular thereto in a rotational movement.

The protective cap described is designed to be compatible with respect to commercially available needle stick protection devices and, in particular, to be adapted to such devices. By means of the protective cap, mechanical impact protection and sealing of syringes embedded in a needle stick protection device can thus be realized at low cost.

In addition, the protective cap enables further beneficial properties. For example, the protective cap contributes to a beneficial labelling capability by increasing a labellable area of the needle stick protection device. This can accommodate more text or larger printing and provide further functionality, such as security solutions or anti-counterfeiting protection, via a larger label. This may also include RFID functionality.

Preferably, the RFID functionality is located in the area of the protective cap or cap body to be localized as far as possible from the liquid in the syringe to minimize potential interference with the RFID functionality by the liquid. For example, an RFID chip and an antenna structure coupled thereto are arranged in or on the cap element. Alternatively, the RFID functionality may be provided in the region of the cap arm or cap arms. In particular, an RFID functionality may be provided in cooperation with a label.

For example, the RFID functionality may be used to enable an electronically readable first opening indication. If a conductive path of the antenna structure in a label or at a predetermined breaking point is damaged when the protective cap is removed, this can be detected by the RFID chip. In addition, the RFID functionality can be used in connection with product tracking, for example in the field of medication management in hospitals.

According to a further aspect of the present invention, a syringe safety system comprises a needle stick protection device for a syringe and an embodiment of the protective cap described above, which is attached to the needle stick protection device by means of the first cap arm and the at least one coupling element. The syringe safety system thus realizes a combination of a needle stick protection device and a protective cap attached thereto.

According to a further aspect of the invention, a syringe system comprises a syringe having a syringe housing, a syringe needle and a needle guard enclosing the syringe needle, and the syringe safety system previously described. The needle stick protection device is coupled to the syringe housing by means of a syringe coupling, so that the protective cap encloses the needle stick protection device. The syringe system thus realizes a combination of a needle stick protection device and a protective cap attached to it, which are coupled to the syringe.

In that the syringe safety system and the syringe system comprise an embodiment of the protective cap described, where applicable, described properties and features of the protective cap are also disclosed for the syringe safety system and the syringe system, and vice versa.

In the following, embodiments of the invention are explained with reference to schematic drawings. They show:

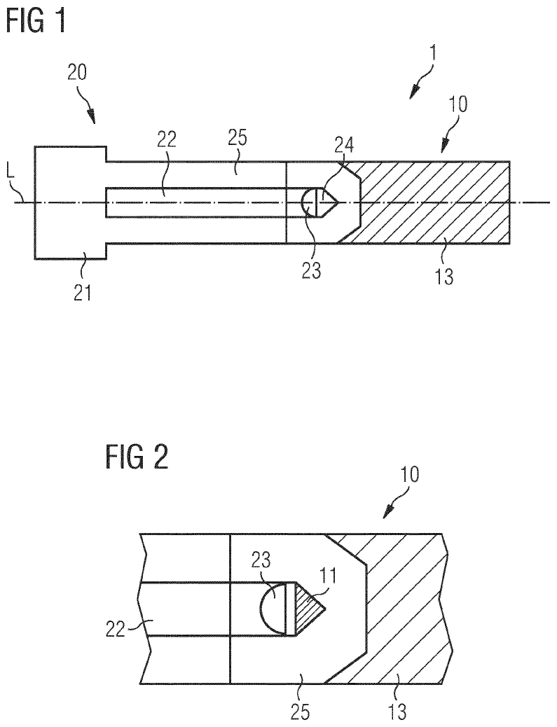

FIGS. 1-5 various embodiments of a protective cap for attachment to a needle stick protection device for a syringe,

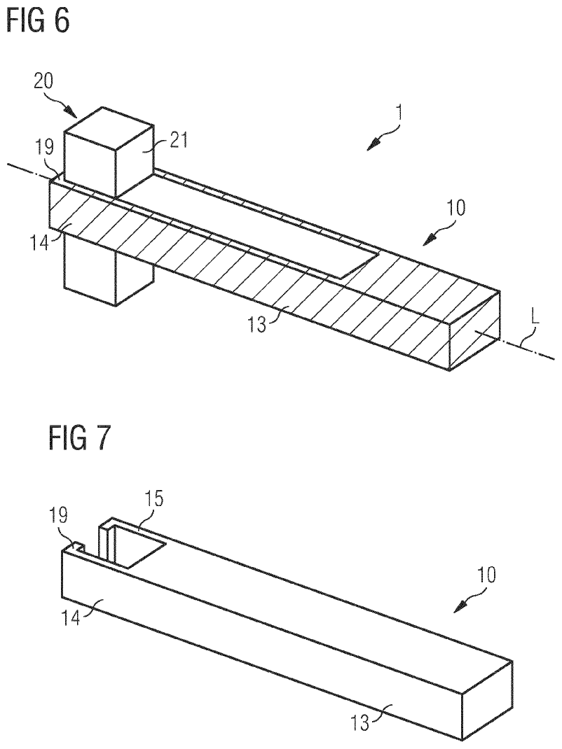

FIGS. 6-9 further embodiments of the protective cap for attachment to a needle stick protection device for a syringe,

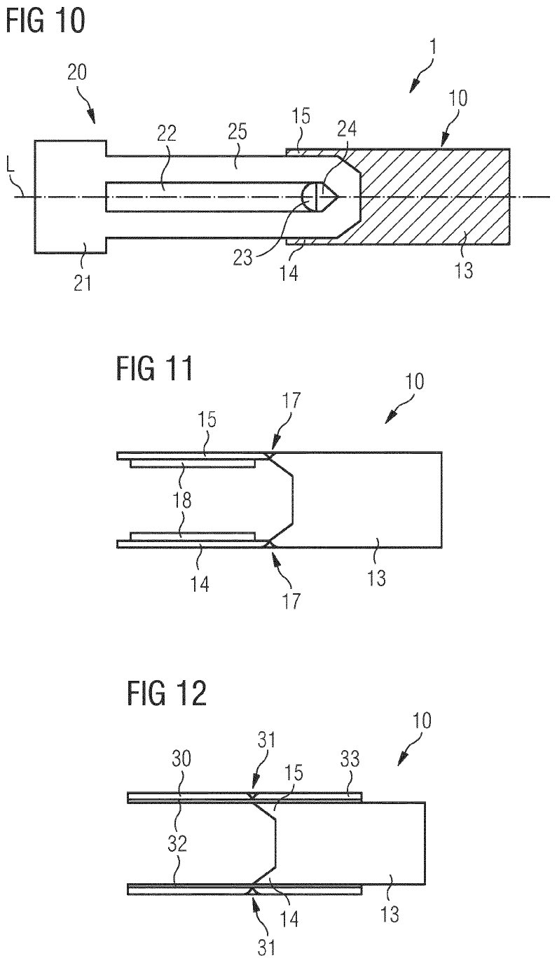

FIGS. 10-12 further embodiments of the protective cap for attachment to a needle stick protection device for a syringe,

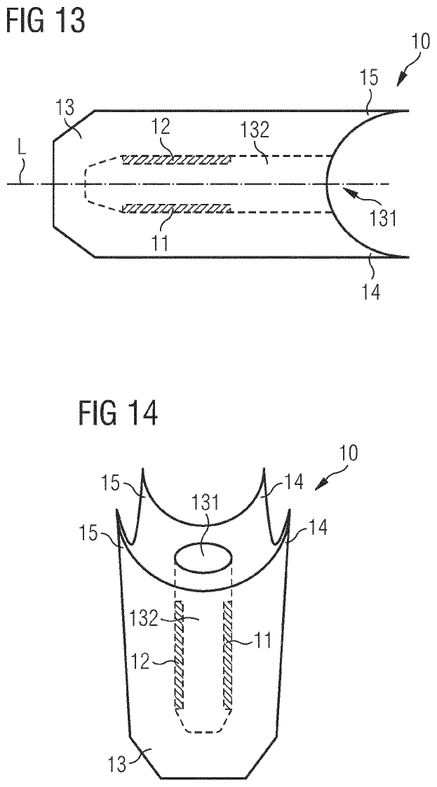

FIGS. 13-15 a further embodiment of the protective cap for attachment to a needle stick protection device for a syringe,

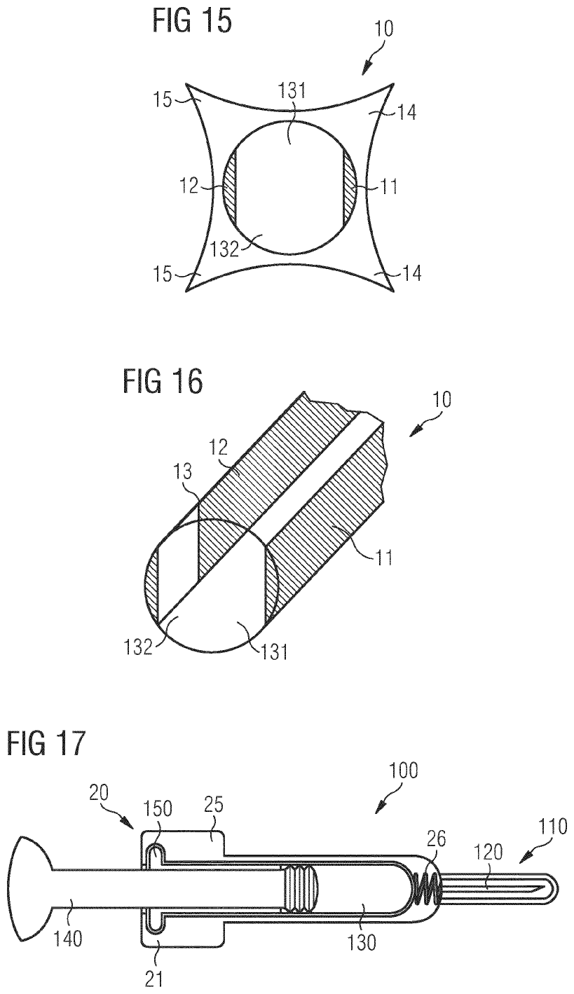

FIG. 16 a further embodiment of the protective cap for attachment to a needle stick protection device for a syringe, and

FIG. 17 a schematic of a conventional syringe.

Elements or features of the same construction or function are marked with the same reference signs throughout the figures. For reasons of clarity, possibly not all elements or features shown in all figures are marked with the corresponding reference signs.

FIGS. 1 to 5 each shows a schematic illustration of embodiments of a protective cap 10 which is designed to be attached to a needle stick protection device 20 for a syringe 100. As will be explained below with reference to FIGS. 1 to 17, the protective cap 10 can be used to provide reliable protection for a syringe 100 provided with the needle stick protection device 20 in a simple and cost-effective manner.

A schematic construction of a conventional syringe 100 is illustrated in FIG. 17. The syringe 100 has a syringe housing 150 which defines a syringe cavity 130 in which, for example, a liquid to be administered is contained. Attached to the syringe housing 150 is a syringe needle 120 surrounded by a needle guard 110. The needle guard 110 comprises, for example, an elastomer element which is attached to the syringe needle 120 as a kind of rubber sheath. Further, a syringe plunger 140 is provided for propelling the contents in the syringe cavity 130 out of the syringe 100 through the syringe needle 120.

The needle stick protection device 20 is arranged on the syringe 100, which is coupled to the syringe housing 150 by means of a syringe coupling 21. The syringe coupling 21 implements a holding mechanism of the needle stick protection device 20 to the syringe 100. The needle stick protection device 20 serves to provide reliable protection from the syringe needle 120 after a use of the syringe 100.

The needle stick protection device 20 is biased with a spring element 26, which realizes a trigger mechanism of the needle stick protection device 20. In a non-triggered state, the needle stick protection device 20 substantially surrounds the syringe plunger 140 and the syringe cavity 130 so that the syringe needle 120 and the needle guard 110 disposed thereon are freely accessible or not surrounded by the needle stick protection device 20. In a triggered state, for example after use of the syringe 100, the spring force of the spring element 26 drives a part of the needle stick protection device 20 towards the syringe needle 120, so that after advancement of this part of the needle stick protection device 20, the syringe needle 120 is surrounded and unwanted contact with the syringe needle 120 is prevented.

It is a finding in connection with the present invention that the needle guard 110 of a pre-filled syringe 100 with inserted syringe needle 120, which are embedded in the needle stick protection device 20, are susceptible to impact effects. When impacted, for example by dropping and striking the syringe 100, a force is applied to the needle guard 110 which may result in damage to the syringe needle 120, for example bending or damage to the ground finish of the syringe needle 120 due to relative movement of the elastomer of the needle guard 110 relative to the syringe needle 120. Further, an unintentionally applied force may cause the needle guard mechanism to disengage or the retaining mechanism of the needle stick protection device 20 to disengage, causing the syringe 100 to slip out of the needle stick protection device 20.

By means of the protective cap 10, additional protection of the syringe 100 and in particular the syringe needle 120 can be contributed to. The protective cap 10 comprises a sleeve-shaped cap body 13 configured to enclose the needle guard 110 of the syringe 100. The protective cap 10 further comprises a cap arm 14, 15 connected to the cap body 13 (for example illustrated in FIGS. 6, 7 and 10). Further, the protective cap 10 comprises at least one coupling member 11, 12, 18, 19, 30 coupled to the cap arm and configured to couple the cap body 13 to an outer housing 25 of the needle stick protection device 20 and to secure the protective cap 10 to the needle stick protection device 20.

FIGS. 1 to 5 show embodiments of the protective cap 10 in which the at least one coupling element is in the form of an engagement element 11, 12. The protective cap 10 has at least one cap arm 14, 15 on which an engagement element 11, 12 is formed that engages in a guide groove 22 of the needle stick protection device 20. The guide groove 22 is implemented by a recess on the outer housing 25 and serves to guide a guide element 23 of the needle stick protection device 20 for the controlled execution of the release mechanism.

FIG. 1 shows a schematic side view of a syringe safety system 1 for the syringe 100, which comprises the needle stick protection device 20 and the protective cap 10 arranged thereon. The needle stick protection device 20 forms a syringe cover which is intended to cover the syringe needle 120, in particular after use of the syringe 100. The protective cap 10 provides additional protection for the syringe 100, in particular for the syringe needle 120.

According to the embodiment example shown in FIG. 2, the protective cap 10 has an engagement element 11 with a triangular contour on each of the cap arms 14 and 15, which engage in the respective guide groove 22 at a front end 24 with respect to a longitudinal axis L of the needle stick protection device 20 or of the protective cap 10. The needle stick protection device 20 has a respective guide channel 22 on opposite sides. The schematically illustrated structure is therefore to be understood as mirror symmetrical with respect to a central axis of the syringe safety system 1. If the protective cap 10 has two opposing cap arms 14 and 15, the respective illustrated design is to be applied to both cap arms 14 and 15.

The triangular contour of the engagement element 11 creates a locking engagement in the outer housing 25 of the needle stick protection device 20 in the elongated guide groove 22. The engagement element 11 is arranged between a front end of the needle stick protection device 20 and the guide element 23 in the guide groove 22 or is designed to engage in this position. The contour of the engagement element 11 is designed to correspond in particular to the predetermined limiting contour of the guide groove 22. The locking contour of the respective engagement element 11 does not interfere with the critical release mechanism of the needle-stick protection device 20.

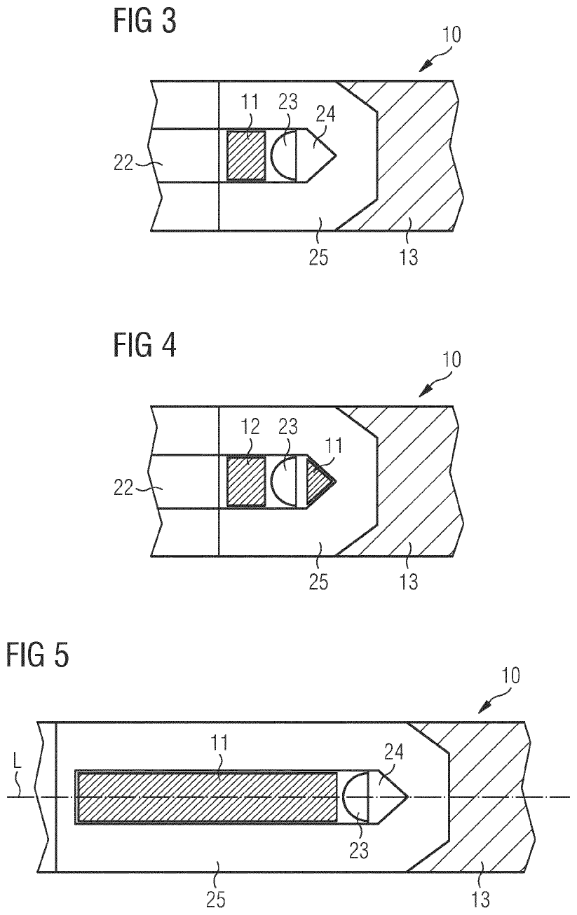

FIG. 3 shows a further embodiment of the protective cap 10 in which the engagement element 11 has a rectangular or square contour and engages in the guide groove 22 behind the guide element 23. In other words, the engagement element 11 engages in the guide groove 22 on an opposite side with respect to the guide element 23 and the front end 24. The predetermined outer contour of the protective cap 10 creates a latching engagement in the outer housing 25 of the needle stick protection device 20 in the elongated guide groove 22, which is commonly provided in conventional needle stick protection devices. The engagement element 11 engages behind the guide element 23 of the needle-stick protection device 20, thus preventing the guide pin or guide element 23 from being blocked and the needle-stick protection mechanism from being triggered unintentionally.

FIG. 4 shows an embodiment of the protective cap 10 in which the cap arm or arms 14, 15 each comprise two engagement elements 11 and 12, one of which engages in the guide groove 22 in front of the guide element 23 and one of which engages in the guide groove 22 behind the guide element 23, with respect to the longitudinal axis L. This embodiment thus represents a combination of the embodiment examples according to FIGS. 2 and 3. Such an embodiment of the protective cap 10 is particularly stable and provides a reliable hold on the needle stick protection device 20. Any load that occurs is absorbed by the protective cap 10 and the outer housing 25. Thus, a locking block of the guide element 23 is not or only slightly mechanically stressed.

FIG. 5 shows a further embodiment of the protective cap 10 in which the one or more cap arms 14, 15 each have an elongated engagement element 11. The elongate engaging contour may beneficially affect a retention of the protective cap 10 on the needle stick protection device 20 and a stability of the syringe safety system 1. In that the elongated engaging element is additionally supported at a rear end or a rear side of the inner contour of the guide groove 22 of the needle stick protection device 20, a defined, stable blocking of the release mechanism of the needle stick protection device 20 is realized. In addition, the extension of the protective cap 10 or the cap arms 14, 15 creates a larger outer surface for additional application possibilities. This can be used in particular for applying one or more labels.

FIGS. 6 to 8 show further embodiments of the protective cap 10 in the form of a clamp or clip component. The protective cap 10 has two cap arms 14 and 15, each of which clasps a portion of the syringe coupling 21 of the needle stick protection device 20 by means of a clamp element 19. The protective cap 10 encloses the outer housing 25 of the needle stick protection device 20 and snaps into place on a rear side of the needle stick protection device 20. Such a large-area or even full-area enclosure of the needle stick protection device 20 can serve, among other things, as UV or light protection in order to shield the contents in the syringe 100 from electromagnetic radiation.

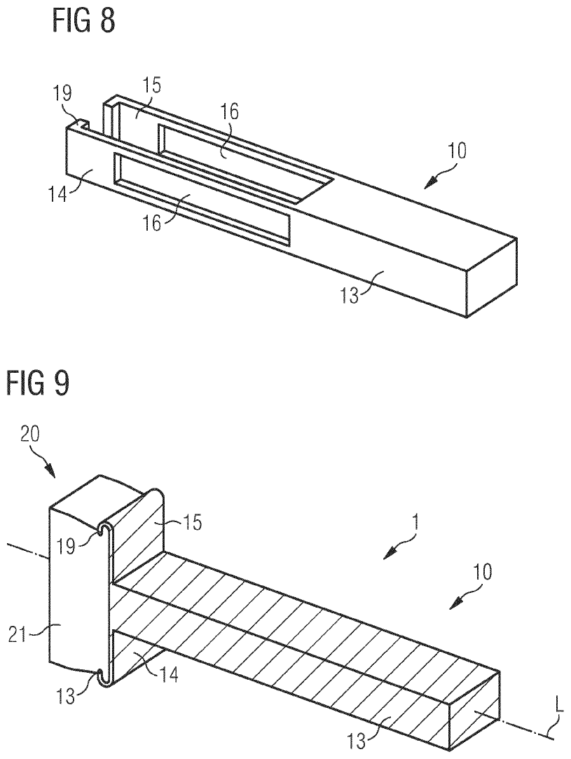

FIG. 8 shows another embodiment of the protective cap 10 in the form of a clip cover, which also has a recess 16 on each of the cap arms 14 and 15. The recesses 16 allow a view through and realize a kind of viewing window for visual inspection of the syringe contents.

FIG. 9 shows a further embodiment example of the protective cap 10 in the form of a clamp sleeve, which by means of the clamp elements 19 respectively clasps a section on an upper side and a lower side of the syringe coupling 21 of the needle stick protection device 20. The clip elements 19 each engage around an edge, a bead or a protruding portion on the syringe coupling 21. The protective cap 10 encloses the outer housing 25 and engages on handle wings of the needle stick protection device 20.

FIGS. 10 to 12 show further embodiments of the protective cap 10, which have coupling elements 18, 30, which in particular enable adhesive attachment of the protective cap 10 to the needle-stick protection device 20.

FIG. 11 shows a schematic side view of the protective cap 10 with the cap arms 14 and 15. An adhesive surface 18 is formed on a respective inner side of the cap arms 14, 15, which enable simple and reliable attachment of the protective cap 10 to the needle stick protection device 20. In addition, the protective cap 10 has a predetermined breaking point 17 between each of the cap arms 14 and 15 and the cap body 13, which can further act as a respective hinge.

Such a protective cap 10 with joint and predetermined breaking point 17 realizes a first-opening protection, which is fixed to the needle stick protection device 20 by means of the adhesive surfaces 18. The predetermined breaking point 17 is irreversibly destroyed by a mechanical movement. The cap body 13 of the protective cap 10 can be removed as a bonnet to expose the syringe needle 120. The cap arms 14 and 15 remain on the needle stick protection device 20 and serve as proof that the syringe 100 or the syringe safety system 1 coupled to the syringe 100 has been opened.

In FIG. 12, a label 30 implements the coupling element of the protective cap 10 to connect it to the needle stick protection device 20. The label 30 has one or more layers and is attached to the cap body 13 by means of an adhesive surface 32. The adhesive surfaces 32 extend beyond the cap body 13 on an underside of the label 32 or on a side facing the cap body 13 to form the cap arms 14 and 15. Where appropriate, as indicated in FIG. 12, relatively short cap arms 14 and 15 are formed so that a respective portion of the label 30 extending away from the cap body 13 realizes an extension of the cap arms 14 and 15. A respective portion of the adhesive surfaces 32 is thus available for adhering the protective cap 10 to the needle puncture protection device 20.

The label 30 further comprises a perforation 31 which allows a predetermined severing of the label 30. The perforation 31 forms a kind of hinge and a predetermined breaking point, so that the protective cap 10 realizes a first-opening protection, which is fixed to the needle stick protection device 20 via the adhesive surfaces 32 of the label 30 with perforation 31.

During an opening process of the syringe 100 or of the syringe safety system 1, the label 30 tears in a predetermined manner along the perforation 31 and the cap body 13 is removed from the needle stick protection device 20 together with a label section 33.

In an alternative embodiment, a label 30 without perforation may also be provided. During an opening operation, such a label nevertheless tears and a label portion 33 is removed from a part of the label remaining on the needle stick protection device 20.

Thus, when opened, the label 30 is irreversibly destroyed by a mechanical movement. The cap body 13 can be removed with the label section 33 like a bonnet. The label 30 can also have other functions, such as forgery protection, UV protection or light protection against visible light and/or RFID functionality.

The mechanical movement for opening the syringe safety system 1 may be a pulling movement or a rotating movement. Further, opening may be by a folding motion, in which case preferably an upper side or outer side of the protective cap 10 is exposed so that the syringe needle 120 is not blocked by the protective cover of the protective cap 10.

In a case the protective cap 10 has two separate cap arms 14 and 15, the respective illustrated embodiment is to be applied to both cap arms 14 and 15. In addition, it is possible that, for example, the first cap arm 14 comprises an engagement element 11, 12, while the second cap arm 15 is connectable to the needle stick protection device 20 by means of an adhesive surface 18, a label 30 and/or a clip element 19.

FIGS. 13 to 16 show further embodiments of the protective cap 10, which comprises coupling elements in the form of engaging or interlocking elements 11, 12 that can interlock with an outer surface of the needle guard 110. The interlocking elements 11 and 12 are, for example, in the form of elongate cuboid shaped projections extending on opposite sides relative to the longitudinal axis L on an inner wall of a cap channel 132. The protective cap 10 further comprises a cap opening 131 which forms an entrance into the cap channel 132 and allows the protective cap 10 to be placed on or over the needle guard 110.

FIG. 14 shows a perspective view of the protective cap 10 shown in FIG. 13. The protective cap 10 has a total of four tapered cap arms 14, 15 which extend from the cap body 13. FIG. 15 shows a view from above or in view of the direction of the cap opening 131 or into the cap channel 132.

FIG. 16 illustrates a further embodiment of the protective cap in a schematic perspective view. The protective cap 10 is comparable to the embodiment example according to FIGS. 13 to 15, but in contrast thereto has no cap arms. The coupling elements 11 and 12 are formed on the inner wall delimiting the cap channel 132 and enable interlocking with the needle guard 110.

By means of the protective cap 10, additional protection is provided for the needle guard 110 and the syringe needle 120. The protective cap 10 is preferably configured to take up or continue a contour of the needle stick protection device 20 and to couple flush with the needle stick protection device 20. Such an embodiment contributes to a useful labelling capability of the syringe safety system 1, so that the protective cap 10 can be easily and reliably connected to the needle stick protection device 20 by means of one or more adhesive labels. When the protective cap 10 is removed to expose the syringe needle 120 during use, irreversible damage to the label 30 and/or the protective cap 1 is preferably purposefully set up so that a proof of first opening can be displayed.

The protective cap 10 for the needle stick protection device 20 provides improved protection for the syringe 100 without having to modify a conventional needle stick protection device. The protective cap 10 avoids transmission of forces acting on the needle stick protection device 20 due to an impact, which may result in bending of the syringe needle 120, damage to a ground section of the syringe needle 120, inadvertent triggering of the needle protection mechanism, or loosening of the holding mechanism of the needle stick protection device 20. The protective cap 10 may further be mechanically interlocked with the needle guard 110 or decoupled from the needle guard 110.

### REFERENCE SIGNS

1 syringe safety system
10 protective cap
11 first engagement element of the protective cap
12 second engagement element of the protective cap
13 sleeve-shaped cap body of the protective cap
131 cap opening
132 cap channel
14 first cap arm of the protective cap
15 second cap arm of the protective cap
16 recess of the cap body
17 hinge/breaking point of the protective cap
18 adhesive surface of the protective cap
19 clamp element of the protective cap
20 needle stick protection device
21 syringe coupling of the needle stick protection device
22 guide groove of the needle stick protection device
23 guide element of the needle stick protection device
24 front end of the guide channel
25 outer housing of the needle stick protection device
26 spring element of the needle stick protection device
30 label
31 perforation of the label
32 adhesive surface of the label
33 removable label section
100 syringe
110 needle guard
120 syringe needle
130 syringe cavity
140 syringe plunger
150 syringe housing
L longitudinal axis of the protective cap/needle guard

1. A protective cap (10) for attachment to a needle stick protection device (20) for a syringe (100), comprising:
a sleeve-shaped cap body (13) adapted to enclose a needle guard (110) of the syringe (100), and
at least one coupling member (11, 12, 18, 19, 30) coupled to the cap body (13) and adapted to couple the cap body (13) to an outer housing (25) of the needle stick protection device (20) and to secure the protective cap (10) to the needle stick protection device (20).

2. The protective cap (10) according to claim 1, comprising: at least one cap arm (14, 15) connected to the cap body (13), wherein at least one coupling element (11, 12, 18, 19, 30) is coupled to the cap arm (14, 15) and is configured to couple the cap body (13) by means of the cap arm (14, 15) to the outer housing (25) of the needle stick protection device (20) and to fasten the protective cap (10) to the needle stick protection device (20).

3. The protective cap (10) according to claim 1, wherein at least one coupling element is formed as an engagement element (11, 12) and is configured to engage in a recess (22) of the needle stick protection device (20).

4. The protective cap (10) according to claim 3, wherein the recess (22) of the needle stick protection device (20) is delimited in a predetermined manner by the outer housing (25) and wherein an outer contour of the engagement element (11, 12) is formed in coordination with a contour delimiting the recess (22) of the needle stick protection device (20).

5. The protective cap (10) according to claim 1, wherein at least one coupling element is formed as a clasping element (19) and is configured to clasp a portion of the needle stick protection device (20).

6. The protective cap (10) according to claim 1, wherein at least one coupling element is formed as an adhesive surface (18) and is configured to form an adhesive coupling with the outer housing (25) of the needle stick protection device (20).

7. The protective cap (10) according to claim 1, wherein at least one coupling element is formed as a label (30) and is configured to connect the cap body (13) to the needle stick protection device (20).

8. The protective cap (10) according to claim 7, wherein the label (30) comprises a perforation (31) along which the label (30) is predeterminedly severable and a label portion (33) is removable together with the cap body (13) from the needle stick protection device (20).

9. The protective cap (10) according to claim 2, comprising:
a predetermined breaking point (17) formed between the cap body (13) and the at least one cap arm (14, 15) such that the protective cap (10) is predeterminedly severable and the cap body (13) is removable from the cap arm (14, 15).

10. The protective cap (10) according to claim 1, comprising:
an interlocking structure formed on an inner surface of the cap body (13) and configured to interlock with an outer surface of the needle guard (110) of a syringe (100) when the protective cap (10) is attached to the syringe (100).

11. The protective cap (10) of claim 1, comprising:
a penetrating recess (16) formed on the cap body (13) and/or the cap arm (14, 15).

12. The protective cap (10) according to claim 1, comprising:
a predetermined weakening structure (17, 31) arranged such that, with respect to a condition arranged on the needle stick protection device (20), an irreversible geometrical change of the protective cap (10) is formed during a removal operation.

13. The protective cap (10) according to claim 1, wherein an outer contour of the protective cap (10) is formed in coordination with an outer contour of the outer housing (25) of the needle stick protection device (20), so that a flush transition between the protective cap (10) and the outer housing (25) of the needle stick protection device (20) can be established with respect to a state arranged on the needle stick protection device (20).

14. The protective cap (10) according to claim 1, comprising:
an RFID chip and an antenna structure coupled to each other and arranged on or in the cap body (13) of the protective cap (10).

15. A syringe safety system (1) comprising:
a needle stick protection device (20) for a syringe (100), and
a protective cap (10) according to claim 1, which is attached to the needle stick protection device (20) by means of the at least one coupling element (11, 12, 18, 19, 30).

16. A syringe system comprising:
a syringe (100) having a syringe housing (150), a syringe needle (120), and a needle guard (110) enclosing the syringe needle (120); and
a syringe safety system (1) according to claim 15, which is coupled to the syringe housing (150) by means of a syringe coupling (21) of the needle stick protection device (20), such that the protective cap (10) encloses the needle guard (110).
